(19) United States
(12) Patent Application Publication
(10) Pub. No.: US 2022/0080218 A1
(43) Pub. Date: Mar. 17, 2022

(54) DEVICE FOR THE TREATMENT OF MALARIA

(71) Applicants: Carmenza SPADAFORA, Panama City (PA); THE PENN STATE RESEARCH FOUNDATION, Pennsylvania, PA (US)

(72) Inventors: Carmenza SPADAFORA, Panama City (PA); Jose Antonio STOUTE, Hershey, PA (US)

(21) Appl. No.: 17/458,101

(22) Filed: Aug. 26, 2021

Related U.S. Application Data
(63) Continuation of application No. PCT/IB2020/051554, filed on Feb. 24, 2020.

(30) Foreign Application Priority Data
Feb. 27, 2019 (GB) 1902634.3

(51) Int. Cl. A61N 5/02; A61N 5/04; A61K 45/06; A61P 33/06

(52) U.S. Cl. CPC A61N 5/022; A61P 33/06; A61K 45/06; A61N 5/04

(57) ABSTRACT

Device for the treatment of malaria A method and device for the treatment of malaria which comprises applying microwave energy to a patient under non-thermal conditions. The non-thermal conditions are achieved by applying the microwave energy under conditions that the normal heating effects of microwaves are not significant. For example, microwave energy may be switched on in pulses lasting less than 6 seconds.

### CROSS-REFERENCE TO RELATED APPLICATIONS

This application is a continuation of PCT/IB2020/051554, filed Feb. 24, 2020, for DEVICE FOR THE TREATMENT OF MALARIA, which claims the benefit of priority to GB Patent Application No. 1902634.3, filed 27 Feb. 2019, both of which are incorporated herein by reference in their entireties.

### FIELD

This invention relates to a device for the treatment of malaria and, in particular, a system for the treatment of malaria using electromagnetic energy in the microwave spectrum.

### BACKGROUND

Malaria is an infectious disease widespread in many tropical and subtropical regions. Plasmodium falciparum, the causative agent of the most devastating form of malaria, causes serious public health problems worldwide. According to the World Health Organization (Fact Sheet: World Malaria Report 2016), in 2015 there were 212 million new cases of malaria worldwide and an estimated 429,000 malaria deaths worldwide. To date, the most effective ways to control malaria have been the use of drugs to treat the disease and insecticides to control the transmission.

Due to the spread of resistance to both types of molecules, there is a need for a new safe and effective treatment for malaria.

The present invention relates to the use of microwave energy in the treatment of malaria.

Microwave irradiation is known to be used in the treatment of cancer, by means of thermal ablation. It has previously been suggested that microwave thermal ablation could also be used to treat malaria. [American Journal of Tropical Medicine and Hygiene (2009), 81(5), Suppl. 1, 44-45. 58th Annual Meeting of the American Society of Tropical Medicine and Hygiene, ASTMH, Washington, Del., United States]

However, thermal ablative therapy, for example when used for the treatment of cancer, does carry risks. The high temperatures produced in microwave thermal ablation can cause collateral thermal damage in the human body, including hemorrhage. The treatment can also cause abscess formation.

It has now been surprisingly found that the non-thermal use microwave energy significantly inhibits the growth of Plasmodium falciparum, without affecting mammalian cells, and is therefore useful in the treatment of malaria.

### SUMMARY

The present invention provides a method for the treatment of malaria which comprises applying microwave energy to a patient, sufficient to reduce the level of malarial parasites in the patient under non-thermal conditions.

### BRIEF DESCRIPTION OF THE DRAWING

FIG. 1 is a schematic representation of a device according to this invention.

### DETAILED DESCRIPTION

In a further aspect, the invention provides a device for the treatment of malaria in a patient, comprising a microwave generator, an applicator connected to the microwave generator and a control means adapted to cause the generator to provide pulses of microwave energy to the patient through the applicator sufficient to reduce the level of malarial parasites in the patient under non-thermal conditions.

The term ‘treatment’ when used herein includes prevention and prophylaxis.

The non-thermal use of microwaves means that the tissues of the patient subjected to microwave energy do not significantly rise in temperature. In particular, the method and device of this invention may comprise the application of microwave energy under conditions wherein the temperature of the tissue of a patient does not exceed 50° C. Suitably, the temperature does not exceed 45° C., preferably not in excess of 42° C.

The non-thermal conditions may be achieved by applying the microwave energy in sufficiently short pulses, so that the normal heating effects of microwaves are not significant. For example, microwave energy may be switched on in pulses lasting less than 6 seconds, suitably from 3 to 5 seconds, preferably about 5 seconds.

Following each pulse of microwave energy, the energy is switched off for an interval before the next pulse. The interval allows the tissues to cool between pulses of microwave energy. The period of the interval should however be sufficiently short to allow continuity in the application of microwave energy.

The period of each cycle, comprising one pulse and one interval, is generally called a duty cycle, and is expressed in terms of the pulse period as percentage of the overall cycle. In the present invention, it is suitable to use from a 10% duty cycle to a 25% duty cycle. The duration of each duty cycle may be, for example, from 15 seconds to one minute, suitably from 20 seconds to 40 seconds. Thus, the interval between pulses may be from 10 to 50 seconds, suitably from 15 to 40 seconds, preferably from 18 to 30 seconds.

Each cycle is then successively repeated to provide a treatment period sufficient to allow the microwave energy to reduce the level of malarial parasites. The treatment period is suitably from 20 minutes to one hour, for example 20 minutes to 50 minutes, preferably from 25 minutes to 45 minutes. During the treatment period, there may be multiple cycles of pulse/interval. For example, there may be from 25 to 150 cycles in one treatment period. It is convenient to have from 50 to 120 cycles per treatment period.

The part of the electromagnetic spectrum defined as microwaves have frequencies from 300 MFIz to 300 GFIz. In this invention, frequencies within this range can be used, in particular from 300 MFIz to 200 GFIz, for example from 300 MFIz to 100 GFIz. It is suitable to use the conventional microwave frequency of 2.45 GHz, which is often chosen because it falls within the band (2.4 to 2.5 GHz) that is not reserved for communication purposes. However, other frequencies may also be used.

The device of this invention comprises a microwave generator, an applicator connected to the microwave generator and a control means. The purpose of the applicator is to transmit the microwaves to the patient. It is possible to connect one applicator, or a plurality of applicators to the microwave generator. For example, two or three applicators may be connected. One suitable type of applicator is a waveguide resonator, comprising an enclosure or cavity, into which the patient's body, or part thereof, is inserted. Waveguides are well known in the art and may be of a size and shape suitable to accommodate the patient's body or part thereof. The waveguide should be closed at least on one side. Alternatively, the applicator may be an open delivery system such an antenna, or a transmission line. The applicator may comprise a ceramic cap, to apply the microwave energy directly onto or into the tissues. Preferably the tip of the applicator is designed for single use to reduce the risk of cross-infection.

Because the malaria parasites are present in the blood, the microwave energy may be applied to any suitable part of the patient's body, for example the torso, arms, legs, hands or feet. Alternatively, the applicator may be in the form of a probe or needle to provide the microwave energy within the body or into a vein.

The connection between the microwave generator and the applicator may be a simple cable, such as a coaxial cable, or may contain further components, such as an amplifier.

The control means is adapted to cause the generator to provide pulses of microwave energy. In its simplest form, the control means may comprise a simple mechanical switch system, which may be manual or programmable. Suitably, the control means is programmable, either mechanically or preferably an electronically, such as in the form of a computer. The control means is generally programmed to provide pre-determined sequence of pulses and interval periods suitable for the treatment. In addition, the control means may also be used to control other variables of the treatment, such as treatment period, frequency and power of the microwave energy.

The control means may be attached to the microwave generator or situated in the connection between the microwave generator and the applicator.

The microwave energy power employed in this invention is such to prevent the patient's body temperature to rise significantly. Suitably the power may be from 3 to 8 Watts.

The above-mentioned parameters of power, pulse times, interval times and treatment times may be varied to maintain non-thermal conditions and achieve the most effective reduction in the level of malarial parasites in the patient.

Patients may preferably be treated in two or more sessions, for example at daily intervals or every other or every third day.

The level of malarial parasites in the blood can be measured by known techniques, by microscopy or by flow cytometry. Parasite species that affect humans are Plasmodium falciparum, Plasmodium vivax, Plasmodium malariae, Plasmodium ovale and Plasmodium knowiesi.

The temperature of the tissues may be measured by a temperature monitoring sensor or probe.

Although this invention is not limited by mechanism of action, our data suggest that the non-thermal microwave energy targets the food vacuole leading to release of intracellular calcium and acidification of the cytoplasm; an opening of calcium channels plays a critical role in this process. There is also a contribution of autophagy in the death of the parasites treated with non-thermal microwave energy.

Our data also show that the microwave treatment according to this invention induces morphological changes that are visible under light microscopy and detectable by flow cytometry, with an increase in reactive oxygen species and lipid peroxidation generation followed by a decrease in cytoplasmic pH, caspase activation and DNA fragmentation.

Furthermore, we have also found that the ability of the parasite P. falciparum to synthesize haemozoin after treatment with microwave energy is not affected. That is, the non-thermal microwave treatment of this invention differs in the mechanism of action of known antimalarials such as chloroquine and mefloquine.

Thus, the use of non-thermal microwave non-microwave energy will act in synergy with antimalarial substances.

Accordingly, in a further aspect of this invention, there is provided an antimalarial substance for use in a method for the treatment of malaria which comprises administering the antimalarial substance to a patient and applying microwave energy to the patient, sufficient to reduce the level of malarial parasites in the patient under non-thermal conditions.

Because the non-thermal microwave energy acts by different mechanism from most antimalarial substances, the microwave energy may be applied prior to, concomitant with, or subsequent to, the administration of the antimalarial substance. Preferably, however, the microwave energy is applied subsequent to the administration of the antimalarial substance.

The antimalarial substance may be a known antimalarial substance, or a substance subsequently found to possess antimalarial activity. For example, the antimalarial substance may be: an aryl amino-alcohol compound, such as quinine, quinidine, mefloquine, halofantrine, lumefantrine, piperaquine, or tafenoquine; an amino-quinoline such as chloroquine, primaquine, or amodiaquine; an antifolate compound, such as pyrimethamine, proguanil, chlorproguanil, trimethoprim, a sulphone or sulphonamide; an artemisinin compound or derivative such as artemisinin, dihydroartemisinin, artemether, artesunate or artelinic acid; a naphthoquinone, such as atovaquone; an iron chelating agent such as desferrioxamine; or an antimicrobial with antimalarial activity, such as tetracycline, doxycycline, clindamycin, azithromycin, or fluoroquinolones. The antimalarial substance may also be a combination of compounds, such as atovaquone in combination with proguanil.

The antimalarial substance may be administered by a route that is suitable to allow absorption of, such as orally, intravenously, intramuscularly, subcutaneously, sublingually, buccally, rectally, vaginally, nasally, topically or transdermally.

Prior to, concomitant with, or subsequent to administration of the antimalarial substance, the non-thermal microwave energy is applied to the patient, as described herein.

One embodiment of the present invention will now be described with reference to the accompanying drawing.

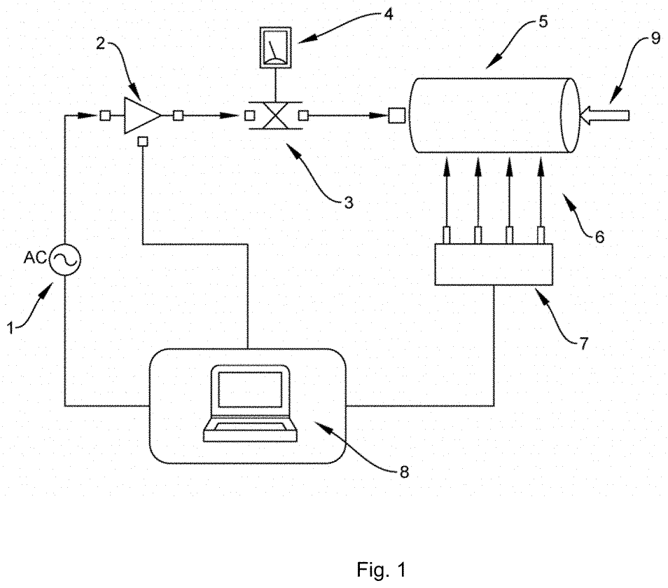

FIG. 1 is a schematic representation of a device according to this invention. A signal generator 1 provides a microwave signal, which is transmitted to a signal amplifier 2. The signal then passes through a coupler 3 having a power indicator 4. The coupler 3 is connected to a waveguide applicator 5, operating at 2.45 GHz. Temperature sensors are connected to an optical fibre thermometer 7. The microwave generator 1, amplifier 2, and thermometer 7 are all connected to a control means in the form of a computer 8.

In operation, a body part, such as a limb, head, neck or torso of a patient suffering from malaria is introduced into the waveguide 5 in the direction shown by the arrow 9. The temperature sensors 6 are attached to the patient. The software on the computer 8 allows a user to control the exposure parameters of pulse and interval times, as well as monitor the temperature, to ensure a non-thermal irradiation.

The invention is further illustrated by in vitro data generated in the following Example.

### Example 1

Experimental Methods

(a) Parasites and Cultures

The malaria parasite strain HB3 of P. falciparum was cultivated following the method described by Haynes et al. [Culture of human malaria parasites Plasmodium falciparum. Nature 263, 767-9 (1976).] with some modifications. In brief, we used 0+ erythrocytes in complete medium that consisted of RPMI 1640 supplemented with 25 mM HEPES, 0.2% sodium bicarbonate, and 10% serum. Blood was obtained from donors under a human protocol approved by the local human use committee. Cultures were maintained at 37° C. in a gas mixture of 5% CO2, 5% O2 and 90% N2 at 2% hematocrit and synchronized with alanine and thermal cycling as described by Almanza et al [Automated Synchronization of P. falciparum using a Temperature Cycling Incubator. Curr Trends Biotech Pharm 1130-1133 (2011).].

(b) Microwave Exposure System

The microwave exposure system consisted of a signal generator, a signal amplifier, a power reflectometer, a waveguide applicator and four optical fiber probes connected to a digital thermometer. All were connected to a computer equipped with custom software BioEMC (Bio Electro Magnetic Compatible) which allowed the user to control all of the exposure parameters as well as store the results of each experiment.

(c) Microwave Exposure Conditions

Experiments were conducted at a frequency of 2.45 GHz. For the resonant cavity, 150 ul of 0.9% saline was placed at the bottom of a 17×100 mm polypropylene tube (Karter Scientific). A small styrofoam holder was placed on top of the saline and a 250 ul polypropylene tube (KeL Scientific KS-3858-WC25) with 200 ul of blood sample was placed in the holder.

The cavity was tuned to always obtain the lowest reflected power and thus maximize the absorption of energy by the sample.

(d) Growth Assays

Following exposure of duplicate samples containing trophozoite or early schizont-synchronized cultures with 2% parasitemia were seeded in 96-well plates for 72 hrs. The growth was monitored for from 24 hrs to up to 72 hrs by microscopy or by flow cytometry. For microscopy, Giemsa-stained thin smears were prepared and the number of infected erythrocytes in at least 1,000 erythrocytes was counted. The microscopist was always blinded to the experimental treatment of each sample. For flow cytometry, samples were stained with 2 pg/ml Hoechst 33342 (Invitrogen, Carlsbad, Calif., USA) for 15 min prior to transferring a 125 ul aliquot to 125 ul of 4% paraformaldehyde in PBS, Samples were stored at 4° C. until acquisition. A CyFlow Space cytometer (Partec, Gorlitz, Germany) was used for acquisition by exciting the samples with a UV laser. The data were analyzed with the FloMax version 2.7 (Quantum Analysis GmbH, Munster, Germany). The change in growth was calculated as (24 h parasitemia−O h parasitemia)/24 h parasitemia.

The results are shown in Table 1.

TABLE 1

| | | | |
|---|---|---|---|
| | | | parasite |
| Irradiation | Pulse on | interval | survival |
| time | (seconds) | (seconds) | (%) |

Power 12 W

| | | | |
|---|---|---|---|
| 20 | 5 | 40 | 80.45 |
| minutes | 5 | 30 | 78.98 |
| | 5 | 25 | 78.5 |
| | 5 | 18 | 65.5 |
| | 5 | 15 | 65.67 |
| 25 | 5 | 40 | 75.35 |
| minutes | 5 | 30 | 77.45 |
| | 5 | 25 | 54.4 |
| | 5 | 18 | 47.2 |
| | 5 | 15 | 50.35 |
| 30 | 5 | 40 | 47.36 |
| minutes | 5 | 30 | 46.21 |
| | 5 | 25 | 37.56 |
| | 5 | 18 | 38.45 |
| | 5 | 15 | 35.15 |
| 45 | 5 | 40 | 18.55 |
| minutes | 5 | 30 | 18.45 |
| | 5 | 25 | 17 |
| | 5 | 18 | 12 |
| | 5 | 15 | 13.5 |

Power 10 W

| | | | |
|---|---|---|---|
| 20 | 5 | 40 | 100 |
| minutes | 5 | 30 | 100 |
| | 5 | 25 | 97.85 |
| | 5 | 18 | 98.78 |
| | 5 | 15 | 98.75 |
| 25 | 5 | 40 | 100 |
| minutes | 5 | 30 | 100 |
| | 5 | 25 | 96.35 |
| | 5 | 18 | 93.86 |
| | 5 | 15 | 93.5 |
| 30 | 5 | 40 | 95.5 |
| minutes | 5 | 30 | 93.45 |
| | 5 | 25 | 94.36 |
| | 5 | 18 | 88.78 |
| | 5 | 15 | 87.9 |
| 45 | 5 | 40 | 87.56 |
| minutes | 5 | 30 | 88.45 |
| | 5 | 25 | 83.9 |
| | 5 | 18 | 77.5 |
| | 5 | 15 | 77.2 |

1. A device for the treatment of malaria in a patient, comprising a microwave generator, an applicator connected to the microwave generator and a control means adapted to cause the generator to provide pulses of microwave energy to the patient through the applicator sufficient to reduce the level of malarial parasites in the patient under non-thermal conditions.

2. A device as claimed in claim 1 wherein the temperature of the tissue of a patient is maintained less than 42 degrees Celsius.

3. A device as claimed in claim 1 wherein microwave energy is switched on in pulses lasting less than 6 seconds.

4. A device as claimed in claim 1 wherein the interval between pulses is from 18 to 30 seconds.

5. A device as claimed in claim 1 wherein the treatment period is from 25 minutes to 45 minutes.

6. A device as claimed in claim 1 wherein the applicator is a waveguide.

7. An antimalarial substance for use in a method for the treatment of malaria which comprises administering the antimalarial substance to a patient and applying microwave energy to the patient, sufficient to reduce the level of malarial parasites in the patient under non-thermal conditions.

8. A method for the treatment of malaria which comprises applying microwave energy to a patient, sufficient to reduce the level of malarial parasites in the patient under non-thermal conditions.

9. A method as claimed in claim 8 wherein the temperature of the tissue of a patient does not exceed 42 degrees Celsius.

10. A method as claimed in claim 8 wherein microwave energy is switched on in pulses lasting less than 6 seconds.

11. A method as claimed in claim 8 wherein the interval between pulses is from 18 to 30 seconds.

12. A method as claimed in claim 8 wherein the treatment period is from 25 minutes to 45 minutes.
